(19) United States
(12) Patent Application Publication
(10) Pub. No.: US 2021/0361359 A1
(43) Pub. Date: Nov. 25, 2021

(54) SYNCHRONIZED TRACKING OF MULTIPLE INTERVENTIONAL MEDICAL DEVICES

(71) Applicant: KONINKLIJKE PHILIPS N.V., EINDHOVEN (NL)

(72) Inventors: RAMON QUIDO ERKAMP, SWAMPSCOTT (NL); HENDRIK ROELOF STAPERT, EINDHOVEN (NL); GUNTHER LAMPARTER, EINDHOVEN (NL); AMEET KUMAR JAIN, BOSTON, MA (US); ALVIN CHEN, CAMBRIDGE, MA (US); SHYAM BHARAT, ARLINGTON, MA (US); KUNAL VAIDYA, BOSTON, MA (US); FRANCOIS GUY GERARD MARIE VIGNON, ANDOVER, MA (US)

(21) Appl. No.: 15/733,902

(22) PCT Filed: Jun. 11, 2019
(86) PCT No.: PCT/EP2019/065093

§ 371 Date: Dec. 1, 2020

Related U.S. Application Data
(60) Provisional application No. 62/685,308, filed on Jun. 15, 2018.

(51) Int. Cl. A61B 34/20

(52) U.S. Cl. CPC A61B 34/20; A61B 2017/3413

(57) ABSTRACT

A controller for determining orientation of an interventional medical device includes a memory that stores instructions, and a processor that executes the instructions. When executed by the processor, the instructions cause the controller to execute a process that includes controlling emission, by an ultrasound probe, of multiple beams each at a different combination of time of emission and angle of emission relative to the ultrasound probe. The process also includes determining, based on receipt of a response to a subset of the multiple beams at a sensor at a location on the interventional medical device, the combination of time of emission and angle of emission relative to the ultrasound probe of one of the subset of the multiple beams. The process also includes determining orientation of the interventional medical device based on the time of emission and angle of emission relative to the ultrasound probe of the one the subset of the multiple beams.

### BACKGROUND

Use of needle procedures under guidance of 2D or 3D probes such as ultrasound probes is widespread and growing. Such procedures may include biopsies, ablations, anesthesia and more. Currently, a tool (e.g., a needle) equipped with a single ultrasound sensor can be used to track location of the tip of the tool but no information about the projected path of the tool. Knowledge of the projected path can help improve workflow and prevent unwanted damage to sensitive anatomical structures. Orientation of the tool is one aspect of information that is useful in projecting a path of the tool. Orientation of a tool may be the relative physical position of the tool, and may be based on or include the shape of the tool including a front, rear, back, sides, top, bottom, and other geometric aspects of the tool. In the case of a tool such as a needle, the shape of the tool may include a shaft as a body, and a tip at the front that may be oriented generally towards the projected path of the tool.

Ultrasound tracking technology estimates the position of a passive ultrasound sensor (e.g., PZT, PVDF, copolymer or other piezoelectric material) in the field of view (FOV) of a diagnostic ultrasound B-mode image by analyzing the signal received by the passive ultrasound sensor as imaging beams from an ultrasound probe sweep the field of view. A passive ultrasound sensor is an acoustic pressure sensor, and these passive ultrasound sensors are used in “InSitu” mechanisms to determine location of the passive ultrasound sensor. Time-of-flight measurements provide the axial/radial distance of the passive ultrasound sensor from an imaging array of the ultrasound probe, while amplitude measurements and knowledge of the direct beam firing sequence provide the lateral/angular position of the passive ultrasound sensor.

FIG. 1 illustrates a known system for tracking an interventional medical device using a passive ultrasound sensor. In FIG. 1, an ultrasound probe 102 emits an imaging beam 103 that sweeps across a passive ultrasound sensor 104 on a tip of an interventional medical device 105. An image of tissue 107 is fed back by the ultrasound probe 102. A location of the passive ultrasound sensor 104 on the tip of the interventional medical device 105 is provided as a tip location 108 upon determination by a signal processing algorithm. The tip location 108 is overlaid on the image of tissue 107 as an overlay image 109. The image of tissue 107, the tip location 108, and the overlay image 109 are all displayed on a display 100.

Guided waves have been used in the field of non-destructive testing (NDT) to determine properties of materials. The properties of a waveguide, the surrounding medium, frequency and angle of insonification determine the occurrence of guided waves.

As suggested above, knowledge of orientation of an interventional medical device 105 helps a clinician see a projected path which can help prevent unwanted rupturing of tissue and provide ways of re-orienting the interventional medical device 105 to circumvent obstacles, thereby improving workflow. However, use of the passive ultrasound sensors 104 to determined orientation requires multiple passive ultrasound sensors 104 and uses the imaging beams from the ultrasound probe 102 that directly impact the passive ultrasound sensors 104.

### SUMMARY

According to an aspect of the present disclosure, a controller for determining orientation of an interventional medical device includes a memory that stores instructions and a processor that executes the instructions. When executed by the processor, the instructions cause the controller to execute a process that includes controlling emission, by an ultrasound probe, of multiple beams each at a different combination of time of emission and angle of emission relative to the ultrasound probe. The process executed by the controller also includes determining, based on receipt of a response to a subset of the multiple beams at a sensor at a location on the interventional medical device, the combination of time of emission and angle of emission relative to the ultrasound probe of one of the subset of the multiple beams. The process further includes determining orientation of the interventional medical device based on the time of emission and angle of emission relative to the ultrasound probe of the one of the subset of the multiple beams.

According to another aspect of the present disclosure, a method for determining orientation of an interventional medical device includes controlling, by a controller that includes a processor that executes instructions, emission by an ultrasound probe of multiple beams each at a different combination of time of emission and angle of emission relative to the ultrasound probe. The method also includes determining, by the controller and based on receipt of a response to a subset of the multiple beams at a sensor at a location on the interventional medical device, the combination of time of emission and angle of emission relative to the ultrasound probe of the one of the subset of the multiple beams. The method further includes determining, by the processor, orientation of the interventional medical device based on the time of emission and angle of emission relative to the ultrasound probe of the one of the subset of the multiple beams.

According to another aspect of the present disclosure, a system for determining orientation of an interventional medical device includes a sensor, an ultrasound probe, and a controller. The sensor is at a location on the interventional medical device. The ultrasound probe emits multiple beams each at a different combination of time of emission and angle of emission relative to the ultrasound probe. The controller includes a memory that stores instructions and a processor that executes the instructions. When executed by the processor, the instructions cause the controller to execute a process that includes controlling emission by the ultrasound probe of the multiple beams. The process also includes determining, based on receipt of a response to a subset of the multiple beams at the sensor, the combination of time of emission and angle of emission relative to the ultrasound probe of the one of the subset of the multiple beams. The process further includes determining orientation of the interventional medical device based on the time of emission and angle of emission relative to the ultrasound probe of the one of the subset of the multiple beams.

### BRIEF DESCRIPTION OF THE DRAWINGS

The example embodiments are best understood from the following detailed description when read with the accompanying drawing FIG.s. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a known system for interventional medical device tracking using a passive ultrasound sensor, in accordance with a representative embodiment.

FIG. 2A illustrates an ultrasound system for relative device orientation determination, in accordance with a representative embodiment.

FIG. 2B illustrates another ultrasound system for relative device orientation determination, in accordance with a representative embodiment.

FIG. 3 is an illustrative embodiment of a general computer system, on which a method of relative device orientation determination can be implemented, in accordance with a representative embodiment.

FIG. 4 illustrates a process for relative device orientation determination, in accordance with a representative embodiment.

FIG. 5 illustrates another process for relative device orientation determination, in accordance with a representative embodiment.

FIG. 6 illustrates production of a guided wave, and a resultant manifestation of the guided wave in relative device orientation determination, in accordance with a representative embodiment.

FIG. 7 illustrates geometry of an interventional medical device operation in relative device orientation determination, in accordance with a representative embodiment.

FIG. 8 illustrates results of pre-calibration of an interventional medical device with different orientations in relative device orientation determination, in accordance with a representative embodiment.

FIG. 9 illustrates charts of guided wave amplitudes as a function of incident angle on an interventional medical device, in accordance with a representative embodiment.

FIG. 10 illustrates aperture variation on beams in relative device orientation determination, in accordance with a representative embodiment.

FIG. 11 illustrates geometry for relative device orientation determination, in accordance with a representative embodiment.

FIG. 12 illustrates geometry of another interventional medical device operation in relative device orientation determination, in accordance with a representative embodiment.

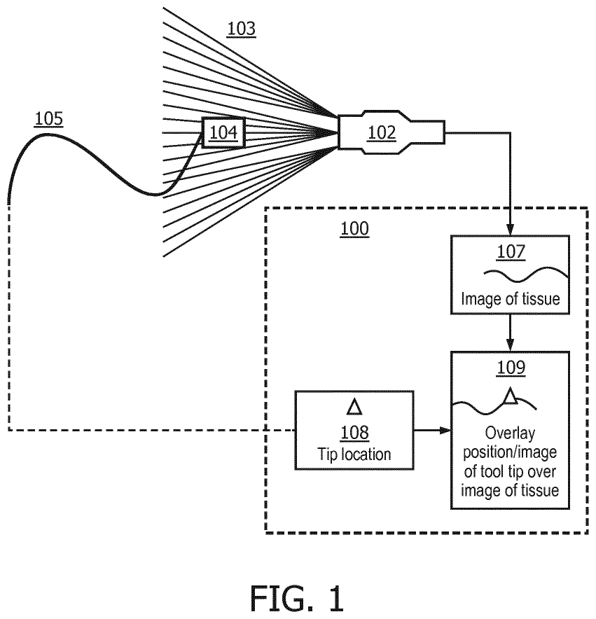

### DETAILED DESCRIPTION

In the following detailed description, for purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only, and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept(s) described herein.

The terminology used herein is for purposes of describing particular embodiments only, and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In view of the foregoing, the present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described below, relative device orientation determination can include determining an angle of insonification that induces guided waves in a medical interventional device. The guided waves and corresponding angle of insonification can then be used to calculate the orientation angle based on pre-determined characteristics of guided waves in a similar interventional medical device in testing.

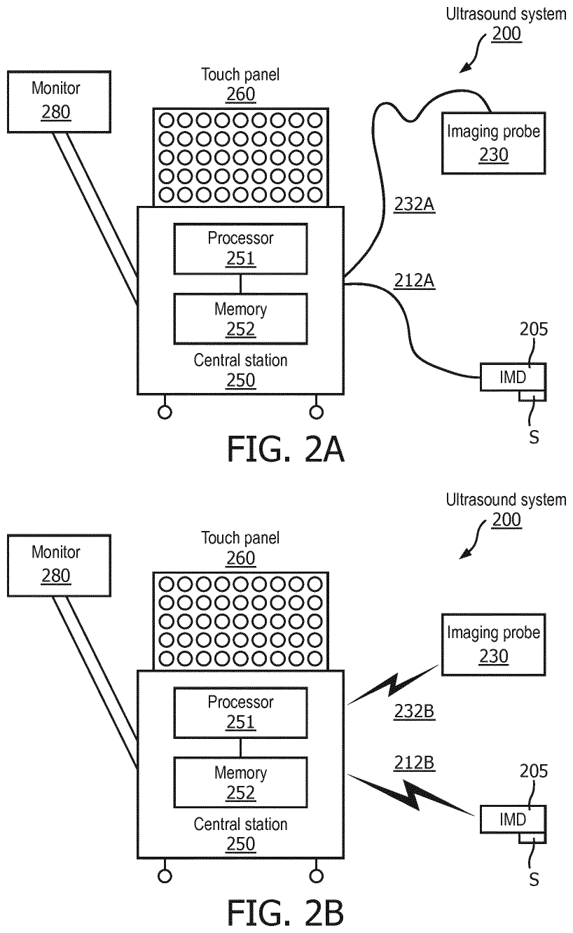

FIG. 2A illustrates an ultrasound system for relative device orientation determination, in accordance with a representative embodiment.

In FIG. 2A, an ultrasound system 200 includes a central station 250 with a processor 251 and memory 252, a touch panel 260, a monitor 280, an imaging probe 230 connected to the central station 250 by wire 232A, and an interventional medical device 205 (IMD) connected to the central station 250 by wire 212A. The imaging probe 230 is an ultrasound probe. A passive ultrasound sensor S is fixed to the interventional medical device 205, though the passive ultrasound sensor S may be fixed to one portion of the interventional medical device 205 and movable relative to another portion of the interventional medical device 205, such as when the passive ultrasound sensor S is fixed to a wire that moves within a sheath. The passive ultrasound sensor S can be, but does not necessarily have to be, provided at an extremity of any portion of the interventional medical device 205.

By way of explanation, the interventional medical device 205 is placed internally into a patient during a medical procedure. Locations of the interventional medical device 205 can be tracked using the passive ultrasound sensor S. The shape of each of the interventional medical device 205 and the passive ultrasound sensor S may vary greatly from what is shown in FIG. 2A and FIG. 2B.

For example, the passive ultrasound sensor S may receive ultrasound tracking beams to help determine a location of the passive ultrasound sensor S. Ultrasound tracking beams described herein may be ultrasound imaging beams that are otherwise used to obtain ultrasound images, or may be ultrasound tracking beams that are separate (e.g., separate frequencies, separate transmission timing) from the ultrasound imaging beams. The passive ultrasound sensor S may be used passively or actively to respond to the received ultrasound tracking beams. As described herein, ultrasound imaging beams and/or ultrasound tracking beams separate from the ultrasound imaging beams can be used to selectively, typically, or always obtain a location of the passive ultrasound sensor S. However, as also noted herein, the tracking can be performed using either or both of the ultrasound imaging beams or completely separate ultrasound tracking beams.

In FIG. 2A, wire 212A and wire 232A are used to connect the interventional medical device 205 and imaging probe 230 to the central station 250. For the imaging probe 230, a wire 232A may not present much of a concern, though the wire 232A may still be a distraction. For the interventional medical device 205, a wire 212A may be used to send back, for example, images when the interventional medical device 205 is used to capture images. However, a wire 212A may be of more concern in that the interventional medical device 205 is at least partly inserted in the patient. Accordingly, replacing the wire 232A and the wire 212A with wireless connections may provide some benefit.

FIG. 2B illustrates another ultrasound system for relative device orientation determination, in accordance with a representative embodiment.

In FIG. 2B, the wire 232A is replaced with wireless data connection 232B, and the wire 212A is replaced with wireless data connection 212B. Otherwise, the ultrasound system 200 in FIG. 2B includes the same central station 250 as in FIG. 2A, i.e., with the processor 251 and memory 252, touch panel 260, monitor 280, imaging probe 230, and interventional medical device 205. The passive ultrasound sensor S moves with the interventional medical device 205.

In FIG. 2B, the ultrasound system 200 may be an arrangement with the interventional medical device 205 with the passive ultrasound sensor S on board. The interventional medical device 205 may include, e.g., a needle with the passive ultrasound sensor S at or near its tip. The passive ultrasound sensor S may also be configured to listen to and analyze data from tracking beams, such that the “sending” of the tracking beams from the imaging probe 230, and the “listening” to the tracking beams by the passive ultrasound sensor S, are synchronized. Use of tracking beams separate from imaging beams may be provided in an embodiment, but not necessarily the primary embodiment(s) of the present disclosure insofar as relative device orientation determination primarily uses embodiments with only imaging beams.

In FIG. 2A or FIG. 2B, the imaging probe 230 may send a pulse sequence of imaging beams. An explanation of the relationship between the central station 250, imaging probe 230 and the passive ultrasound sensor S follows. In this regard, central station 250 in FIGS. 2A and 2B may include a beamformer (not shown) that is synchronized by a clock (not shown) to send properly delayed signals in a transmit mode to elements of an imaging array in the imaging probe 230. In a receive mode, the beamformer may properly delay and sum signals from the individual elements of the imaging array in the imaging probe 230. The ultrasound imaging itself is performed using the imaging probe 230, and may be in accordance with beamforming performed by the beamformer of the central station 250.

The imaging probe 230 may emit imaging beams as tracking beams that impinge on the passive ultrasound sensor S (i.e., when the passive ultrasound sensor S is in the field of view of the tracking beams). The passive ultrasound sensor S may receive and convert the energy of the tracking beams into signals so that the passive ultrasound sensor S, the interventional medical device 205, the imaging probe 230 or the central station 250 can determine the position of the passive ultrasound sensor S relative to the imaging array of the imaging probe 230. The relative position of the passive ultrasound sensor S can be computed geometrically based on the received tracking beams received by the passive ultrasound sensor S, and the relative position can be used to identify orientation of the interventional medical device 205 as it is deployed in a patient. Orientation of the interventional medical device 205 is an angle of orientation of the interventional medical device

As described herein, received tracking beams may be considered direct beams when they directly impact a passive ultrasound sensor S. Direct beams may directly hit the passive ultrasound sensor S, and may be considered a direct wave, but hereinafter will be referred to as direct beams. However, guided waves can also be generated in/on the interventional medical device 205, such as a guided wave that travels down the shaft of a needle. The guided waves are a response to receipt of direct beams, and may uniquely correspond to and identify a “critical” angle in which a direct beam arrives at the interventional medical device 205 and induces generation of the guided wave. The orientation of the interventional medical device 205 can be determined with knowledge of the angle of emission of the direct beam that induces a guided wave (e.g., a guided wave with a highest intensity among guided waves received at a passive ultrasound sensor S), and knowledge of the critical angle that will result in such a guided wave in/on the interventional medical device 205. Thus, in relative device orientation determination, the passive ultrasound sensor S is used to detect a guided wave, and not just direct beams from the imaging probe 230, and the guided wave can be used to identify (isolate) a particular direct beam that induces the guided wave.

Thus, the imaging probe 230 emits tracking beams to the interventional medical device 205 for a period of time that includes multiple different points of time. For example, tracking beams may be emitted for 30 seconds, 60 seconds, 120 seconds, 180 seconds or any other period of time that include multiple different points of time. The tracking beams may be emitted by the imaging probe 230 in an ordered combination of time of emission and angle of emission relative to the imaging probe 230 (ultrasound probe). Energy of the tracking beams (direct beams) and guided waves (each induced by a subset of one or more of the direct beams) may be collected periodically as responses to the direct beams and guided waves, such as every second or every 1/10th second. The responses to the tracking beams may be reflected energy reflected by the passive ultrasound sensor S. Alternatively, the responses to the tracking beams may be active signals generated by the passive ultrasound sensor S, such as readings of the received energy of the tracking beams. The responses to he guided waves are typically based on readings of the received energy of the guided waves.

Based on the responses to the tracking beams, the processor 251 may determine, for example, absolute position of the passive ultrasound sensor S at multiple different points in time during a period of time. Orientation of the interventional medical device 205 may be determined based on knowledge of the critical angle, using one absolute position matched with identification of one direct beam corresponding to one angle of emission, along with receipt of the guided wave corresponding to the one direct beam. As a result, orientation of the interventional medical device 205 can be determined. The specifics of several embodiments for how to identify the one beam are described below in relation to other FIGs.

The central station 250 may be considered a control unit or controller that controls the imaging probe 230. As described in FIGS. 2A and 2B, the central station 250 includes a processor 251 connected to a memory 252. The central station 250 may also include a clock (not shown) which provides clock signals to synchronize the imaging probe 230 with the passive ultrasound sensor S. Moreover, one or more elements of the central station 250 may individually be considered a control unit or controller. For example, the combination of the processor 251 and the memory 252 may be considered a controller that executes software to perform processes described herein, i.e., to use a position of the passive ultrasound sensor S and the direct beam corresponding to a specific angle of emission to determine orientation of the interventional medical device 205 as the interventional medical device 205 is deployed in a patient.

The imaging probe 230 is adapted to scan a region of interest that includes the interventional medical device 205 and the passive ultrasound sensor S. Of course, as is known for ultrasound imaging probes, the imaging probe 230 also uses ultrasound imaging beams to provide images on a frame-by-frame basis. The imaging probe 230 can also use separate tracking beams to obtain the location of the passive ultrasound sensor S.

In a one-way relationship, the passive ultrasound sensor S may be adapted to convert tracking beams provided by the imaging probe 230 into electrical signals. The passive ultrasound sensor S may be configured to provide either the raw data or partially or completely processed data (e.g., calculated sensor locations) to the central station 250, either directly or indirectly (e.g., via a transmitter or repeater located in a proximal end of the interventional medical device 205). These data, depending on their degree of processing, are either used by the central station 250 to determine the location of the passive ultrasound sensor S (and the location of the distal end of the interventional medical device 205 to which the passive ultrasound sensor S is attached), or to provide the central station 250 with the location of the passive ultrasound sensor S (and the location of the distal end of the interventional medical device 205 to which the passive ultrasound sensor S is attached).

As described herein, the positions of the passive ultrasound sensor S are determined by or provided to the central station 250. The positions of the passive ultrasound sensor S can be used by the processor 251 to overlay the positions of the passive ultrasound sensor S and the orientation of the interventional medical device 205 onto an image frame for display on the monitor 280.

Broadly, in operation, the processor 251 initiates a scan by the imaging probe 230. The scan can include emitting imaging beams as tracking beams across a region of interest. The imaging beams are used to form an image of a frame; and as tracking beams to determine the location of the passive ultrasound sensor S. As can be appreciated, the image from imaging beams is formed from a two-way transmission sequence, with images of the region of interest being formed by the transmission and reflection of sub-beams. Additionally, in a one-way relationship, the imaging beams as tracking beams incident on the passive ultrasound sensor S and may be converted into electrical signals (i.e., rather than or in addition to reflecting the tracking beams). In a two-way relationship, the imaging beams as tracking beams are reflected by the passive ultrasound sensor S, so that the imaging probe 230 determines the location of the passive ultrasound sensor S using the reflected tracking beams.

As noted above, data used to determine locations of the passive ultrasound sensor S may be or include raw data, partially processed data, or fully processed data, depending on where location is to be determined. Depending on the degree of processing, these data can be provided to the processor 251 for executing instructions stored in the memory 252 (i.e., of the central station 250) to determine the positions of the passive ultrasound sensor S in the coordinate system of ultrasound images from the beamformer. Alternatively, these data may include the determined positions of the passive ultrasound sensor S in the coordinate system which is used by the processor 251 when executing instructions stored in the memory 252 to overlay the position of the passive ultrasound sensor S and the orientation of the interventional medical device 205 on the ultrasound image in the monitor 280. To this end, the beamformer of the central station 250 may process the beamformed signal for display as an image of a frame. The output from the beamformer can be provided to the processor 251. The data from the passive ultrasound sensor S may be raw data, in which case the processor 251 executes instructions in the memory 252 to determine the positions of the passive ultrasound sensor S in the coordinate system of the image; or the data from the passive ultrasound sensor S may be processed by the passive ultrasound sensor S, the interventional medical device 205, or the imaging probe 230 to determine the locations of the passive ultrasound sensor S in the coordinate system of the image. Either way, the processor 251 is configured to overlay the positions of the passive ultrasound sensor S and the orientation of the interventional medical device 205 on the image on the monitor 280. For example, a composite image from the imaging beams as tracking beams may include the image of tissue and actual or superposed positions of the passive ultrasound sensor S and the orientation of the interventional medical device 205, thereby providing real-time feedback to a clinician of the position of the passive ultrasound sensor S (and the distal end of the interventional medical device 205) and orientation of the interventional medical device 205, relative to the region of interest.

As described with respect to FIG. 2A and FIG. 2B, an ultrasound system for relative device orientation determination can be used to provide orientation of medical devices equipped with a single sensor. Insofar as ultrasound waves striking a needle shaft result in a guided wave propagating through the shaft of the needle at a speed different from the direct beams travelling through tissue, these guided waves can be used to identify when the specific relative angle between the ultrasound direct beam and the needle is the critical angle. Detecting the presence of these guided waves helps determine the needle orientation. Ultrasound direct beams can be fired at multiple angles to induce these guided waves. Detection of a shaft propagated guided wave in response to a beam of a known angle and pre-calibrated data based on rotational directivity of the needle is therefore used to determine the orientation of the needle.

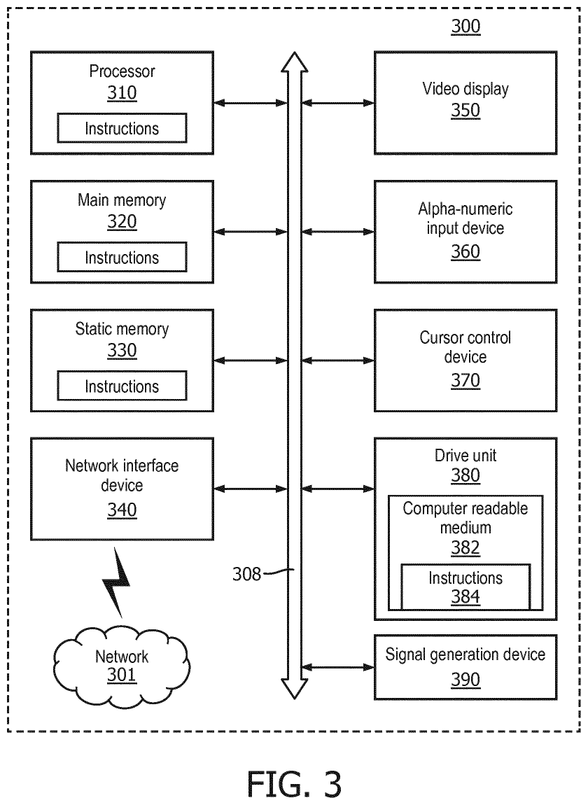

FIG. 3 is an illustrative embodiment of a general computer system, on which a method of relative device orientation determination can be implemented, in accordance with a representative embodiment.

The computer system 300 can include a set of instructions that can be executed to cause the computer system 300 to perform any one or more of the methods or computer based functions disclosed herein. The computer system 300 may operate as a standalone device or may be connected, for example, using a network 301, to other computer systems or peripheral devices.

The computer system 300 can be implemented as or incorporated into various devices, such as a stationary computer, a mobile computer, a personal computer (PC), a laptop computer, a tablet computer, an ultrasound system, an ultrasound probe, a passive ultrasound sensor S, an interventional medical device 205, an imaging probe 230, a central station 250, a controller, or any other machine capable of executing a set of instructions (sequential or otherwise) that specify actions to be taken by that machine. The computer system 300 can be incorporated as or in a device that in turn is in an integrated system that includes additional devices. In an embodiment, the computer system 300 can be implemented using electronic devices that provide voice, video or data communication. Further, while the computer system 300 is illustrated as a single system, the term “system” shall also be taken to include any collection of systems or sub-systems that individually or jointly execute a set, or multiple sets, of instructions to perform one or more computer functions.

As illustrated in FIG. 3, the computer system 300 includes a processor 310. A processor for a computer system 300 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A processor is an article of manufacture and/or a machine component. A processor for a computer system 300 is configured to execute software instructions to perform functions as described in the various embodiments herein. A processor for a computer system 300 may be a general-purpose processor or may be part of an application specific integrated circuit (ASIC). A processor for a computer system 300 may also be a microprocessor, a microcomputer, a processor chip, a controller, a microcontroller, a digital signal processor (DSP), a state machine, or a programmable logic device. A processor for a computer system 300 may also be a logical circuit, including a programmable gate array (PGA) such as a field programmable gate array (FPGA), or another type of circuit that includes discrete gate and/or transistor logic. A processor for a computer system 300 may be a central processing unit (CPU), a graphics processing unit (GPU), or both. Additionally, any processor described herein may include multiple processors, parallel processors, or both. Multiple processors may be included in, or coupled to, a single device or multiple devices.

Moreover, the computer system 300 includes a main memory 320 and a static memory 330 that can communicate with each other via a bus 308. Memories described herein are tangible storage mediums that can store data and executable instructions, and are non-transitory during the time instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. A memory described herein is an article of manufacture and/or machine component. Memories described herein are computer-readable mediums from which data and executable instructions can be read by a computer. Memories as described herein may be random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. Memories may be volatile or non-volatile, secure and/or encrypted, unsecure and/or unencrypted.

As shown, the computer system 300 may further include a video display unit 350, such as a liquid crystal display (LCD), an organic light emitting diode (OLED), a flat panel display, a solid-state display, or a cathode ray tube (CRT). Additionally, the computer system 300 may include an input device 360, such as a keyboard/virtual keyboard or touch-sensitive input screen or speech input with speech recognition, and a cursor control device 370, such as a mouse or touch-sensitive input screen or pad. The computer system 300 can also include a disk drive unit 380, a signal generation device 390, such as a speaker or remote control, and a network interface device 340.

In an embodiment, as depicted in FIG. 3, the disk drive unit 380 may include a computer-readable medium 382 in which one or more sets of instructions 384, e.g. software, can be embedded. Sets of instructions 384 can be read from the computer-readable medium 382. Further, the instructions 384, when executed by a processor, can be used to perform one or more of the methods and processes as described herein. In an embodiment, the instructions 384 may reside completely, or at least partially, within the main memory 320, the static memory 330, and/or within the processor 310 during execution by the computer system 300.

In an alternative embodiment, dedicated hardware implementations, such as application-specific integrated circuits (ASICs), programmable logic arrays and other hardware components, can be constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing can be constructed to implement one or more of the methods or functionality as described herein, and a processor described herein may be used to support a virtual processing environment.

The present disclosure contemplates a computer-readable medium 382 that includes instructions 384 or receives and executes instructions 184 responsive to a propagated signal; so that a device connected to a network 101 can communicate voice, video or data over the network 301. Further, the instructions 384 may be transmitted or received over the network 301 via the network interface device 340.

In out of plane (OOP) procedures, the passive ultrasound sensor S may lie outside of the ultrasound plane, whereas for in plane procedures, the passive ultrasound sensor S can be used for relative device orientation determination in conjunction with an imaging probe 230 that is two-dimensional. In the out of plane procedures, energy from direct beams received by the passive ultrasound sensor S may be under a detectable threshold, and the guided wave response would be the only response that is detectable, and a matrix probe would be required to produce tracking beams in the elevational direct. Moreover, even for in-plane procedures, an interventional medical device 205 may be hard to see in ultrasound-guided procedures, especially at the oblique insertion angles used in most freehand procedures such as soft tissue biopsies, ablations etc. Orientation helps clinicians determine a projected path which can help prevent unwanted rupturing of tissue and provides ways of re-orienting the interventional medical device 205 to circumvent obstacles, thereby improving workflow. The ability to predict the path of the instrument using only one sensor as described herein can reduce costs and may see a high level of acceptance within the clinical community. A computer system 300 may use a processor 310 to process data and instructions, including readings from the passive ultrasound sensor S, predetermined characteristics of the interventional medical device 205 including the known critical angle, and knowledge of the emission timing and emission angles of a sequence of direct beams fired by the imaging probe 230. As a result, orientation of the interventional medical device 205 can be determined, and used to help a clinician project the path of the interventional medical device 205.

Relative device orientation determination provides navigation using an instrument with a single sensor, thereby reducing manufacturing costs. Relative device orientation determination also improves work flow by enabling quicker procedures by virtue of the path prediction. Moreover, relative device orientation determination can help prevent rupture of sensitive anatomical structures by providing knowledge of the projected path the interventional medical device 205.

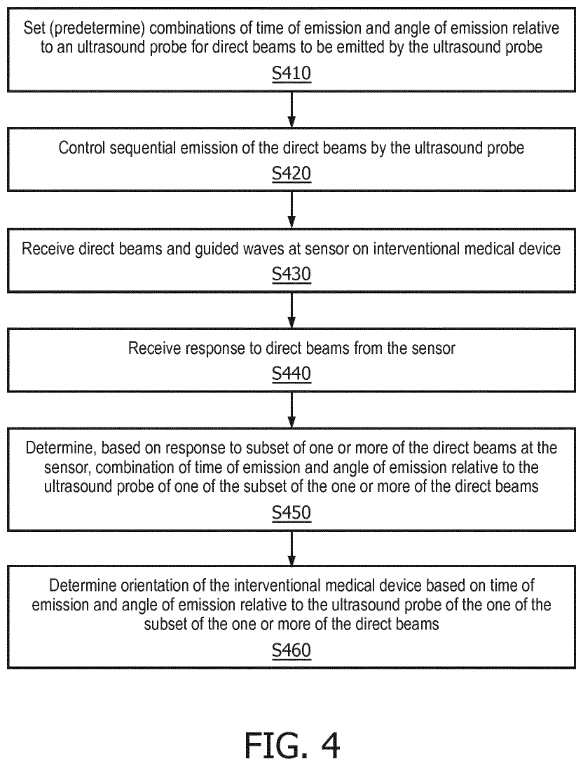

FIG. 4 illustrates a process for relative device orientation determination, in accordance with a representative embodiment.

At S410, combinations of time of emission and angles of emission relative to an ultrasound probe (e.g., imaging probe 230) are set (predetermined) for direct beams to be emitted by the ultrasound probe. These combinations of time of emission and angles of emission can be set for each specific medical device (e.g., interventional medical device 205), and for each different intervention on patients. A resultant response of the medical device can be measured for each combination. In other words, in the process of FIG. 4, medical devices may be pre-characterized by insonification at a range of known angles between the medical devices and direct beams. Testing may involve subjecting different medical devices to a range of dozens, hundreds or even thousands of direct beams from different relative angles, to generate a directivity curve for each medical device. The directivity curves for different medical devices may be stored as a lookup table in a memory, and referenced for dynamic relative device orientation determination when the medical devices are used as the interventional medical device 205. Of course, the specific information of a directivity curve that is most important for any particular medical device is which relative angle results in the highest strength signal (highest intensity) from an induced guided wave, as this is the critical angle described herein. Signal strength corresponding to the guided wave propagated along the shaft of the interventional medical device 205 (e.g., a needle) as a response to a subset of the emitted direct beams is recorded for all the angles in pre-testing, so that this information can be predetermined for each different medical device. In this way, InSitu technology that provides the 2D position of the passive ultrasound sensor S can be used to determine the origin of the direct beams that are fired at a range of known angles to evoke the response based on the guided wave propagating through the shaft. The response to the direct beams is detected and the angle corresponding to the direct beam(s) generating the guided wave propagated along the shaft is recorded. This angle and the pre-determined response of the medical device is used to estimate the orientation. Using the 2D position of the passive ultrasound sensor S on the interventional medical device 205, and the orientation of the interventional medical device 205, the projected path may be rendered on the ultrasound (B-mode) image.

At S420, the sequential emission of the direct beams by the ultrasound probe is controlled. The direct beams may be emitted in a known sequence of dozens, hundreds or thousands of individual direct beams, each in a differentiable combination of time of emission and angle of emission. Additionally, when the ultrasound probe has multiple apertures (which may be true in almost any embodiment), a specific aperture may be specifically selected for each emitted directed beam or set of emitted direct beams. Thus, a complete sequence of direct beams emitted as a result of the control at S420 may be emitted from a single aperture, or from different apertures specifically selected for each direct beam of subset of direct beams.

At S430, the direct beams and guided waves are received at the passive ultrasound sensor S on the interventional medical device 205. As a reminder, the guided waves are a form of response to a subset of one or more direct beams, as is the energy received at the passive ultrasound sensor S from direct impact/receipt of a direct beam. The passive ultrasound sensor S may measure signal strength periodically, such as every 1/10 of a second, every 1/100th of a second, or at the same rate as the rate at which direct beams are emitted. The emission of direct beams as a result of the control at S420 and the receipt of the direct beams and guided waves at the passive ultrasound sensor S may be synchronized indirectly in that each received or measured/detected direct beam or guided wave may be matched with an emitted direct beam based on the logical processes described herein.

At S440, responses to the direct beams are received from the passive ultrasound sensor S. As noted, responses may be measurements of a direct beam that is directly detected by the passive ultrasound sensor S, or measurements of a guided wave that is propagated along the shaft of the interventional medical device 205. The measurements or other characteristics of each detected direct beam or guided wave may be sent from the passive ultrasound sensor S to the ultrasound probe. That is, the responses measured or otherwise detected at the passive ultrasound sensor S can be compared to known characteristics of responses measured or otherwise detected in testing of a similar interventional medical device in testing. The response received at the passive ultrasound sensor S reflecting a highest intensity of a guided wave travelling down the shaft of the interventional medical device 205 may be identified as a response of interest. All measured responses, or fewer than all measured responses, may be compared to the predetermined combinations of time of emission and angles of emission to see which measured responses likely correspond to which particular direct beam. However, the response of interest may be the response with the highest intensity in comparison to responses to other direct beams at other combinations of time of emission and angles of emission. Thus, a response identified as having the highest intensity may be identified by comparing a response to one of the direct beams with responses to others of the direct beams. The response of interest may correspond to a so-called “critical” angle determined in advance, and knowledge of which angel of emission for a beam resulted in the response of interest can be used together with knowledge of the predetermined critical angle to identify the orientation of the interventional medical device 205. The critical angle may be one and only one critical angle of emission among all angles of emission that will result in the response of interest.

Moreover, as explained herein, the guided wave that propagates along the shaft of the interventional medical device 205 (e.g., a needle) may be of specific use insofar as the guided wave may correspond to a particular direct beam, and the difference between the direct beam and the orientation of the interventional medical device 205 may be the critical angle when the guided wave is detected.

At S450, combinations of time of emission and angle of emission relative to the ultrasound probe can be determined based on responses (guided waves and/or energy of an impinging direct beam) to a subset of one or more of the direct beams received at the passive ultrasound sensor S. As noted repeatedly herein, the guided wave that travels along the shaft of the interventional medical device 205 may be of special import, as this guided wave may correspond to a direct beam emitted at a particular relative angle of emission (e.g., the critical angle) that has been predetermined at S410.

At S460, orientation of the interventional medical device 205 is determined based on time of emission and angle of emission of one of the subset of direct beams relative to the ultrasound probe. That is, the predetermined critical angle may be used to determine the relative orientation of the interventional medical device 205, since the angle of emission of the direct beam is identified, the time of emission of the direct beam is identified, and the guided wave that travels along the shaft of the interventional medical device 205 may be of sufficient strength to indicate that the direct beam is emitted at the predetermined critical angle relative to the interventional medical device 205. Thus, when the guided wave that travels along the shaft of the interventional medical device 205 is detected, it can be used to correlate which emitted direct beam caused the guided wave, which in turn can be used with the critical angle to derive the orientation of the interventional medical device 205.

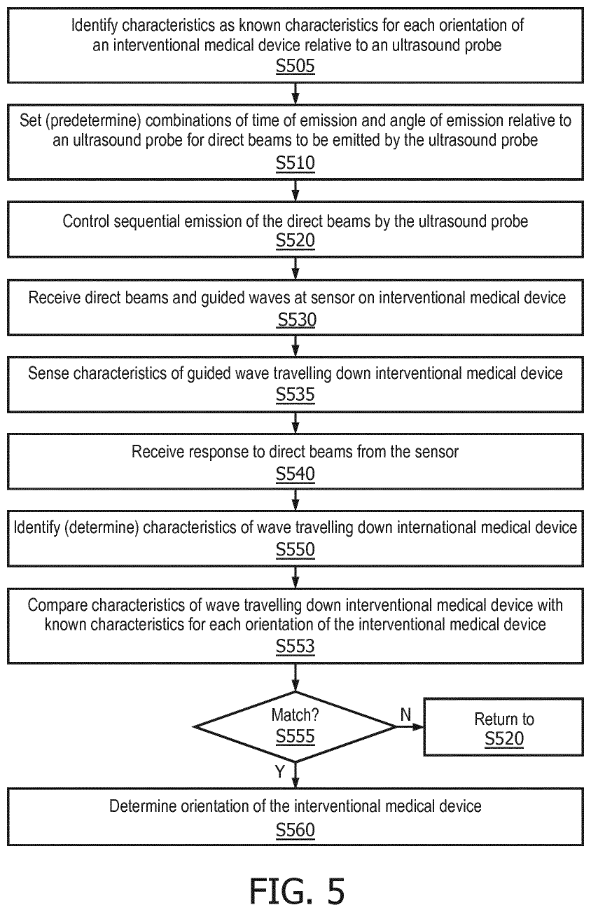

FIG. 5 illustrates another process for relative device orientation determination, in accordance with a representative embodiment.

At S505, characteristics for each orientation of the interventional medical device 205 relative to an ultrasound probe are identified as known characteristics. The identification at S505 may be performed systematically using a testing pattern, such as in a laboratory. The characteristics can be stored as a table of data for each orientation. As noted herein, a characteristic specifically of interest is the critical angle which results in a guided wave propagating down the shaft of the interventional medical device 205, as there may be only one such critical angle which produces the guided wave with the maximum strengths.

At S510, combinations of time of emission and angles of emission relative to an ultrasound probe are set (predetermined) for direct beams to be emitted by the ultrasound probe. That is, an emission pattern may be set in advance so that direct beams are systematically emitted at different angles of emission, such as at predetermined intervals. A resultant response of the medical device can be subsequently measured for each combination. As explained already, medical devices may be pre-characterized by insonification at a range of known angles between the medical devices and ultrasound direct beams. Signal strength corresponding to a guided wave propagated along the shaft of the interventional medical device 205 (e.g., a needle) as a response to a subset of the emitted direct beams is recorded for all the angles. Directivity curves for different medical devices may be generated and stored as a lookup table in memory.

In more detail, using InSitu technology, a 2D position of the passive ultrasound sensor S can be identified. Identification of the emitted direct beam that results in the proper wave propagating down the shaft of the interventional medical device 205 provides the angle of emission of the emitted direct beam. Reference to the predetermined characteristics obtained at S505 provides for the critical angle, which can be used with the angle of emission to identify the orientation of the interventional medical device 205. Using the 2D position of the passive ultrasound sensor S on the interventional medical device 205, and the orientation of the interventional medical device 205, the projected path may then be superimposed on an ultrasound image.

At S520, the sequential emission of the direct beams by the ultrasound probe is controlled, and at S530, the direct beams and guided waves are received at the passive ultrasound sensor S on the interventional medical device 205. Operations at S520 and S530 may be the same or similar to those explained with respect to the corresponding numbered operations in FIG. 4, and descriptions thereof are therefore not repeated.

At S535, characteristics of a guided wave travelling down the interventional medical device 205 are sensed. For example, maximum amplitude of a signal can be measured, time of the maximum amplitude can be recorded, and so on. As noted previously, responses measured by the passive ultrasound sensor S may be measurements of a direct beam that is directly detected by the passive ultrasound sensor S, or measurements of a guided wave that is propagated along the shaft of the interventional medical device 205 and sensed by the passive ultrasound sensor S. The characteristics of interest at S535 are characteristics of the guided wave travelling down the interventional medical device 205. The passive ultrasound sensor S sends the responses to the direct beams, and at S540 the responses to the direct beams are received from the passive ultrasound sensor S at the imaging probe 230.

At S550, characteristics of the guided wave travelling down the interventional medical device 205 are identified, such as at the central station 250. The central station 250 may receive all data readings from the passive ultrasound sensor S, or a limited set based on signals that reach a minimum threshold. A minimum threshold is a predetermined threshold, determined in advance. At S553, the characteristics of the guided wave travelling down the interventional medical device 205 are compared with known characteristics for each orientation of the interventional medical device 205. Alternatively, reference may be made to the known critical angle for the interventional medical device 205, as the different between a particular direct beam with a known angle of emission and the orientation of the interventional medical device 205 may be approximately equal to or identical to the critical angle.

At S555, a determination is made whether a match is found when comparing the characteristics of a guided wave with the known characteristics identified at S505. If no match is found (S555=No), the process returns to S520. If a match is found (S553=Yes), the orientation of the interventional medical device 205 is determined at S560.

As an example of an embodiment consistent with the teachings of FIG. 5, the process includes preparatory processes including determining the critical angle at which the guided wave is strongest for the interventional medical device 205 (e.g., a needle) as a part of a pre-calibration. The preparatory processes include the identification at S505, and may include other processes as described herein. Dynamic processes may include determining an optimal aperture that will be used to insonify the passive ultrasound sensor S at multiple different angles of emission, and then obtaining measurements from the passive ultrasound sensor S to distinguish measurements (a “blob”) corresponding to direct beams from measurements (“blobs) corresponding to the guided wave. Time of flight can be determined from the measurements corresponding to the guided wave and the angle of the corresponding direct beam. The predetermined critical angle can then be used in the dynamic processing, along with identification of the direct beam corresponding to the guided wave with the peak signal intensity, to determine the needle orientation at S560.

As described above, a system for determining orientation of an interventional medical device 205 may include the interventional medical device 205 such as medical equipment equipped with one passive ultrasound sensor S near the tip. Processes may be divided between preparatory processes and dynamic processes. In a preparatory process, different interventional medical devices such as needles can be characterized such that a range of responses (i.e., including guided waves) to beams at multiple angles are known. The responses can include signal intensity/signal strength for each different relative angle used in the preparatory process. A critical angle can then be determined in the preparatory process based on the range of responses, such as by identifying the angle of the direct beam corresponding to the guided wave with the highest signal intensity/signal strength received at the passive ultrasound sensor S. As noted previously, the guided wave travels to the passive ultrasound sensor S before the corresponding direct beam hits the passive ultrasound sensor S. Insofar as the guided wave is a response to receipt of a subset of one ore more direct beams, the time of receipt of the guided wave in a dynamic process can be compared to time of receipt of the corresponding direct beam. The determining in the dynamic process of a combination of time of emission and angle of emission relative to the ultrasound probe of one of the subset of the direct beams corresponding to the critical angle of emission may be selectively performed only when the time of receipt of one response to the direct beams (e.g., the guided wave) is prior to the time of receipt of the other response to the direct beams (i.e., the energy of the direct beam received at the passive ultrasound sensor S). The system may include means to distinguish between a sensor response from a hit by a direct beam and a sensor response from a guided wave, and the means may include a processor that executes software instructions to process information from the passive ultrasound sensor S. The system may also include means to control the elements of the transducer such that steered beams across multiple angles can be fired from a desired aperture. The aperture may be selectively identified in order to optimize one or more angles of the multiple angles across which steered beams are fired.

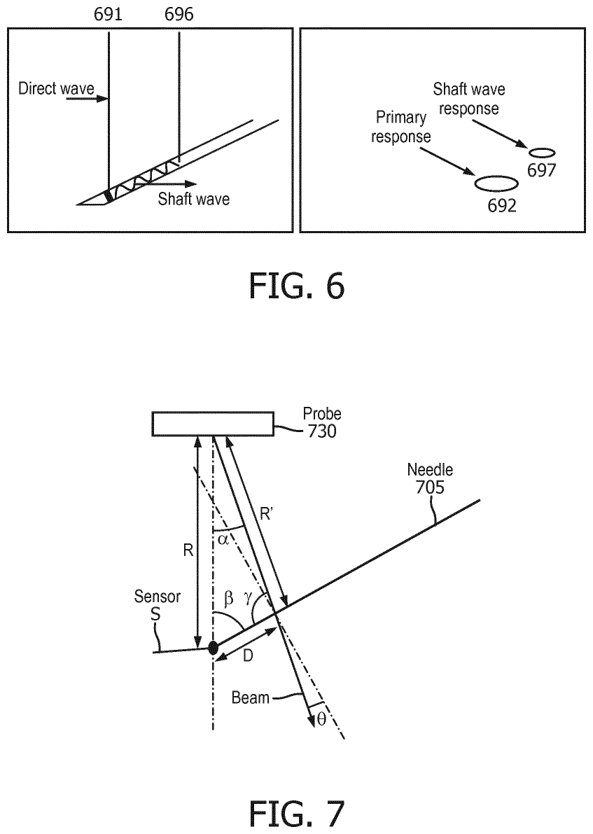

FIG. 6 illustrates production of a guided wave, and a resultant manifestation of the guided wave in relative device orientation determination, in accordance with a representative embodiment.

Guided waves are produced when one of the ultrasound direct beams intercepts an interventional medical device 205 such as a needle shaft and travels through the needle shaft (at a speed greater than that in tissue) to the passive ultrasound sensor S. The guided waves travel down the interventional medical device 205 and have an intensity that is measurable. The guided wave travelling down the interventional medical device 205 having a highest intensity of guided waves generated in response to the direct beams results in identification as the response of interest, and ultimately is used to identify which direct beam at which orientation caused the guided wave with the highest intensity in comparison to responses caused by the other direct beams. As noted previously, knowledge of the critical angle which results in a response (guided wave) with known characteristics may be part of a set of known characteristics determined in advance and corresponding to different orientations of the interventional medical device 205 relative to the ultrasound probe. This response manifests itself before the blob produced by the primary response as shown in the FIG. 6. Since the guided waves are known to be produced at very specific angles between the ultrasound direct beam and the orientation of the interventional medical device 205, knowledge of this critical angle and the origin of the blob can be used to determine the orientation of the interventional medical device 205. The orientation of the interventional medical device 205 is thus an angle of orientation of the interventional medical device 205 determined based on angle of emission relative to the imaging probe 230 of one of a subset of direct beams and the critical angle of the interventional medical device 205.

FIG. 7 illustrates geometry of an interventional medical device operation in relative device orientation determination, in accordance with a representative embodiment.

In FIG. 7, the geometry is used to determine a critical angle in a pre-calibration step. In a preparatory process, calibration can be performed in a controlled water tank experiment where a needle (as an example of an interventional medical device 205) is fixed to a stage which is rotated. The position of the needle is adjusted such that the same ultrasound direct beam insonifies the needle at every rotational position. The sensor response recorded is the combination of direct beams that directly hit the passive ultrasound sensor S after travelling through water and direct beams that hit the shaft of the needle thereby inducing a guided wave at specific angles that travel through the shaft as a surface wave. The data collected can be processed by compensating for the water path traversed by the direct beams through a time offset. Data is reconstructed as a function of the distance the guided wave travels through the needle. Realigning the data after the time offset, the speed of the guided wave travelling through the shaft can be calculated. In one such lab experiment two surface waves were detected. One detected surface wave travels at approximately 3250 m/s and another at 1400 m/s. The faster wave is the guided wave, and reached the passive ultrasound sensor S before the direct beam hits the passive ultrasound sensor S. By thresholding the data of the passive ultrasound sensor S to only allow the response pertaining to the guided wave it is possible to estimate the transmission angle and the needle rotation angle that produces the strongest response amongst the datasets collected.

The needle is insonified in a controlled setup at a range of angles and the response from the passive ultrasound sensor S is recorded. The data of the passive ultrasound sensor S is recomputed to compensate for the path travelled in tissue. The arrival time t (time of arrival) of a guided wave is expressed as a function of the distance travelled in water and the distance travelled in the needle:

$$t = \frac{{R}^{′}}{c} + \frac{D}{{c}_{g}}$$

Where R′ is the distance travelled in water, c is the speed of sound in water, D is the distance travelled in the needle, and c₉ is the speed of the guided wave in the needle.

The law of sines yields

$$D = \frac{R \cdot sin ( α )}{sin ( π - α - β )}$$

Using this formula, the receive trace can be drawn as a function of the distance travelled in the needle, D, for each acquisition (each needle angle β and each beam angle α) after time adjusting the trace using an offset corresponding to the distance travelled in water.

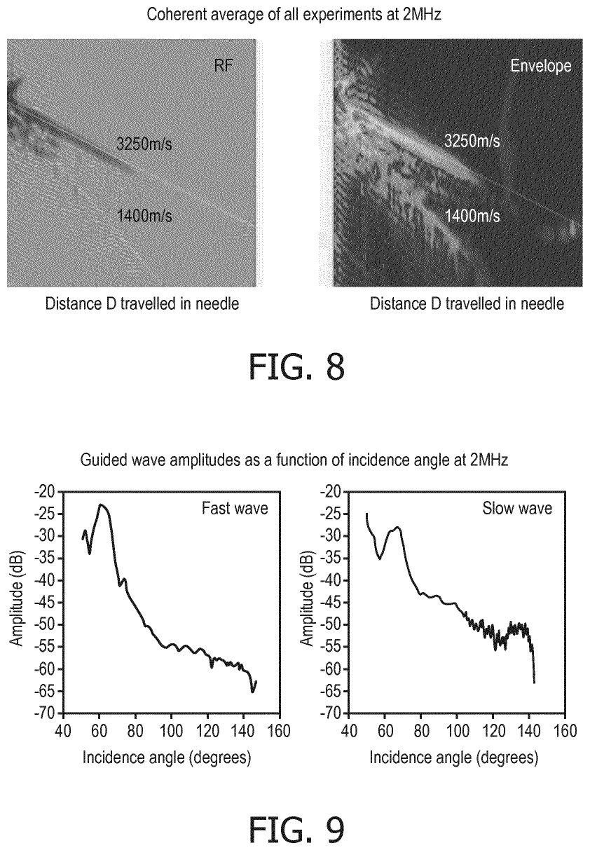

Coherently averaging all the traces from all acquisitions yields FIG. 8, which is representative of such a pre-calibration step.

FIG. 8 illustrates results of pre-calibration of an interventional medical device with different orientations in relative device orientation determination, in accordance with a representative embodiment.

The strength of the guided wave can be estimated as a function of incidence angle γ. Using the law of sines again, the angle γ is known for each experiment (each distance travelled in the needle D) by

$$γ = asin ( sin ( α ) \frac{D}{R} )$$

For all experiments that yield a significant amount of guided wave, a temporal window is drawn around the (fast) guided wave and (slow) direct beam, and the maximum amplitude of the guided wave within this temporal window is recorded, to yield the charts on FIG. 9 as described below.

FIG. 9 illustrates charts of guided wave amplitudes as a function of incident angle on an interventional medical device, in accordance with a representative embodiment.

In FIG. 9, amplitudes of the fast (left) guided wave and slow (right) direct beam at 2 MHz are shown as a function of incidence angle. The fast guided wave peaks at ˜62° and the slow direct beam at ˜69°.

A peak for the fast guided wave is clearly seen at ˜60 degrees incidence angle. Here the incidence angle is defined as the angle between the ultrasound direct beam and the needle, such that 90 degrees would be normal incidence. This is similar to what is commonly observed in the lab with peaks when the needle is ˜30 degrees from the horizontal. A peak for the slow direct beam is seen at ˜70 degrees incidence angle. It is important to note that these results will vary depending on the needle used and the other factors like frequency of ultrasound.

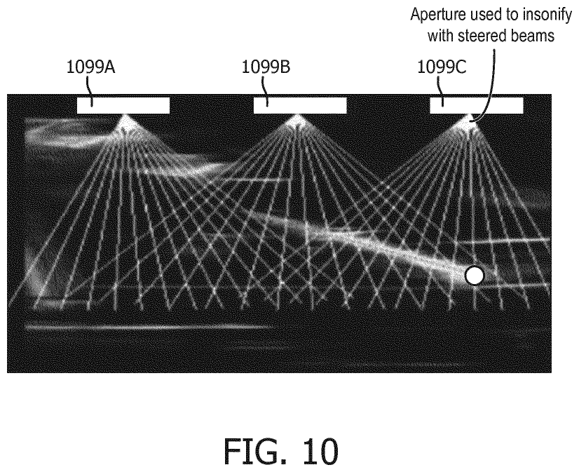

FIG. 10 illustrates aperture variation on beams in relative device orientation determination, in accordance with a representative embodiment.

The InSitu technology is used to estimate the position of the passive ultrasound sensor S as described in the background section. Based on this estimate an appropriate aperture is used to insonify the needle with steered beams at multiple angles such that the needle shaft is exposed to as many beams as possible as shown in FIG. 10.

The location of the needle corresponding to the direct beam is determined using the InSitu method described in the background section. The data of the passive ultrasound sensor S is thresholded using a temporal window to exclude the response from the direct beam(s). The remainder of the data may be or include the response from the guided wave.

A more elaborate method to determine the origin of the response is the estimation of the speed of wave that induced the corresponding blob as described in the pre-calibration step.

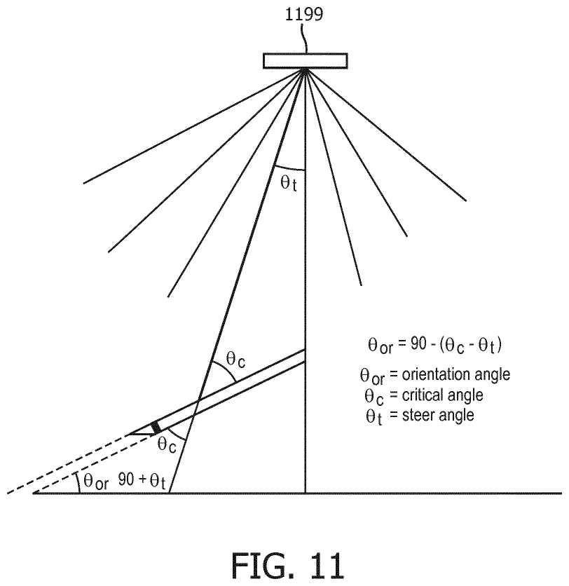

FIG. 11 illustrates geometry for relative device orientation determination, in accordance with a representative embodiment.

In FIG. 11, needle orientation is estimated according to an embodiment. The location of the needle corresponding to the direct beam is determined using the known InSitu method. The data of the passive ultrasound sensor S is thresholded using a temporal window to exclude the response from the direct beam. This data now may be or include the guided wave response. The origin of the peak of this response is traced back to the direct beam that induced it. The angle of the direct beam as shown in FIG. 11 is θₜ. The critical angle θ_{c} is determined using the pre-calibration step. The orientation angle θₒᵣ as shown in FIG. 11 is calculated using the equation θₒᵣ=90−(θ_{c}+θₜ), where θₒᵣ is the orientation angle, θ_{c} is the critical angle, and θₜ is the steer angle.

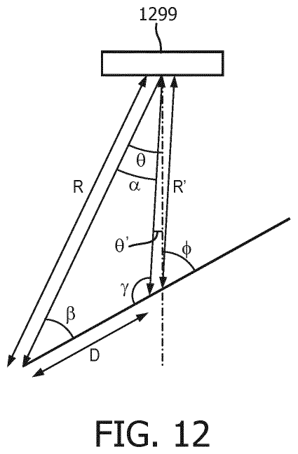

FIG. 12 illustrates geometry of another interventional medical device operation in relative device orientation determination, in accordance with a representative embodiment.

In FIG. 12, an incidence angle of a guided wave can be computed according to an embodiment. In the embodiment of FIG. 12, the position of both the response to the direct beam and the guided wave response is used to arrive at an estimate of the orientation angle. Given (t, θ) and (t′, θ′), and assuming the first blob (t, θ) corresponds to arrival of the direct beam and the second blob (t′, θ′) corresponds to arrival of the guided wave, the incidence angle γ of the direct beam that corresponds to the guided wave can be computed. In the embodiment of FIG. 12, t is the arrival time of the first blob and t′ is the arrival time of the second blob. If the incidence angle γ is close to the value for maximum guided wave generation (˜60°), then it is likely that the second blob is a guided wave.

Al Kashi's law of cosines gives:

D²=R²+R′²−2RR′ cos(α),

where D is the distance travelled in the needle. The arrival time of the second blob is expressed by

$${t}^{′} = \frac{{R}^{′}}{c} + \frac{D}{{c}^{′}} ,$$

where c, c′ are the speeds of the direct beam in the tissue and the guided wave in the needle, respectively. Replacing

$${R}^{′} = c {t}^{′} - \frac{c}{{c}^{′}} D$$

into me first expression above (Al Kashi's law), one obtains a second-order polynomial in D that can be solved for D. Note that R is known from analysis of the first blob, and a is the difference between measured angles in the first and second blobs (α=θ−θ′). Once D is known, the law of sines can be used again to determine the incidence angle γ:

$$sin ( γ ) = \frac{R}{D} sin ( α ) .$$

In another embodiment, a check can be made whether a second blob identified at (t, θ) is the arrival of the direct beam associated with this guided wave. This embodiment assumes that the first blob is identified as (t′, θ′), for example when the first blob is identified using the first arrival algorithm. In this embodiment, the check determines whether the second blob identified at (t, θ) is the arrival of the direct beam associated with this guided wave. In a sense this is the opposite of method described immediately above, and similar geometrical derivations can be made.

Accordingly, relative device orientation determination enables use of a single passive ultrasound sensor S on an interventional medical device 205 such as a needle, cannula or other tracked tool. Relative device orientation determination provides for feedback of the orientation on a user interface such as the monitor 280. Production of orientation information based on a guided wave can be performed in different ways described herein.

Relative device orientation determination can be applied to most areas that make use of sensor based medical devices including but not limited to regional anesthesia for pain management, biopsies, ablations and vascular access procedures. Relative device orientation determination can be performed even with the most challenging anatomy which makes the interventional medical device 205 (e.g., a needle) hardest to see. Additionally, relative device orientation determination can be applied to both 1D and 2D array transducers, and in 2D array transducers the orientation of the medical device in 3D can be estimated.

Although relative device orientation determination has been described with reference to several exemplary embodiments, it is understood that the words that have been used are words of description and illustration, rather than words of limitation. Changes may be made within the purview of the appended claims, as presently stated and as amended, without departing from the scope and spirit of relative device orientation determination in its aspects. Although relative device orientation determination has been described with reference to particular means, materials and embodiments, relative device orientation determination is not intended to be limited to the particulars disclosed; rather relative device orientation determination extends to all functionally equivalent structures, methods, and uses such as are within the scope of the appended claims.

As described above, relative device orientation determination can be accomplished using a single passive ultrasound sensor S, so long as characteristic responses of interventional medical device 205 are determined in advance, including which relative angle between a beam and the interventional medical device 205 will result in a guided wave with a maximum intensity. Additional aspects, such as the ability to distinguish between a guided wave and a direct beam, help improve the accuracy of relative device orientation determination. Additional aspects such as aperture selection can be used to optimize the number of ultrasound direct beams that will directly hit the interventional medical device 205. Moreover, the overall methods described herein may include preliminary steps, such as the determination of characteristic responses for different interventional medical devices, and dynamic steps, such as the dynamic determination of relative device orientation using a passive ultrasound sensor S on an interventional medical device 205 inserted into a patient.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the FIG.s are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

1. A controller for determining an orientation of an interventional medical device, comprising:
a memory that stores instructions; and
a processor that executes the instructions,
wherein, when executed by the processor, the instructions cause the controller to execute a process comprising:
controlling emission, by an ultrasound probe, of a plurality of beams each at a different combination of time of emission and angle of emission relative to the ultrasound probe;
determining, based on receipt of a response to a subset of the plurality of beams at a sensor at a location on the interventional medical device, the combination of time of emission and angle of emission relative to the ultrasound probe of one of the subset of the plurality of beams,
determining the orientation of the interventional medical device based on the time of emission and angle of emission relative to the ultrasound probe of the one of the subset of the plurality of beams, and determining characteristics of a wave travelling down the interventional medical device as the response to the subset of the plurality of beams,
wherein determining the orientation is additionally based on the characteristics of the wave travelling down the interventional medical device as the response to the subset of the plurality of beams.

2. (canceled)

3. The controller of claim 1,
wherein the process executed by the controller further comprises:
comparing the characteristics of the wave travelling down the interventional medical device as the response to the subset of the plurality of beams with a set of known characteristics corresponding to different orientations of the interventional medical device; and
matching the characteristics of the wave travelling down the interventional medical device with one of the set of known characteristics,
wherein determining the orientation is additionally based on the one of the set of known characteristics matched with the characteristics of the wave travelling down the interventional medical device.

4. The controller of claim 3,
wherein the process executed by the controller further comprises:
determining, based on the sensor sensing the characteristics of the wave travelling down the interventional medical device, the combination of time of emission and angle of emission relative to the ultrasound probe of the one of the subset of the plurality of beams.

5. The controller of claim 4, wherein the set of known characteristics corresponding to different orientations of the interventional medical device are determined in advance to identify a critical angle of the interventional medical device which will generate a response with highest intensity to the one of the plurality of beams in comparison to other beams received as the receipt to the response at the sensor.

6. The controller of claim 1,
wherein the response to the subset of the plurality of beams comprises a guided wave travelling down the interventional medical device, and
the guided wave travelling down the interventional medical device has a highest intensity of guided waves generated in response to the plurality of beams, and is generated only in response to the subset of the plurality of beams including a beam at one and only one critical angle of emission among all angles of emission of the plurality of beams.

7. The controller of claim 1,
wherein the sensor is one and only one sensor used to determine location on the interventional medical device.

8. The controller of claim 1,
wherein the process executed by the controller further comprises:
comparing characteristics of a wave travelling down the interventional medical device as the response to the subset of the plurality of beams with a set of known characteristics corresponding to different orientations of the interventional medical device; and
when no match is found between the characteristics of the wave travelling down the interventional medical device and the set of known characteristics, again controlling emission, by the ultrasound probe, of the plurality of beams each at a different combination of time of emission and angle of emission relative to the ultrasound probe, to determine the orientation of the interventional medical device.

9. The controller of claim 1,
wherein the process executed by the controller further comprises:
comparing the response to the subset of the plurality of beams to a predetermined threshold, and only determining the combination of time of emission and angle of emission relative to the ultrasound probe of the one of the subset of the plurality of beams when the response to the subset of the plurality of beams is above the predetermined threshold.

10. The controller of claim 9,
wherein the process executed by the controller further comprises:
comparing a time of receipt of the response to the subset of the plurality of beams to time of receipt of another response to another subset of the plurality of beams, and only determining the combination of time of emission and angle of emission relative to the ultrasound probe of the one of the subset of the plurality of beams based on whether the response to the subset of the plurality of beams is received prior to the time of receipt of the other response to the plurality of beams.

11. The controller of claim 1,
wherein the process executed by the controller further comprises:
calculating a distance through the interventional medical device travelled by the response to the subset of the plurality of beams to the sensor.

12. The controller of claim 1,
wherein the orientation of the interventional medical device is an angle of orientation of the interventional medical device determined based on angle of emission relative to the ultrasound probe of the one of the subset of the plurality of beams.

13. The controller of claim 1,
wherein the process executed by the controller further comprises:
determining the location of the sensor on the interventional medical device,
wherein determining the orientation is additionally based on the location of the sensor on the interventional medical device.

14. The controller of claim 1,
wherein the process executed by the controller further comprises:
determining time of arrival at the sensor of the receipt of the response to the subset of the plurality of beams,
wherein determining the orientation is additionally based on the time of arrival at the sensor of the response to the subset of the plurality of beams which produces a response with the highest intensity compared to other subsets of the plurality of beams.

15. A method for determining an orientation of an interventional medical device, comprising:
controlling, by a controller comprising a processor that executes instructions, emission by an ultrasound probe of a plurality of beams each at a different combination of time of emission and angle of emission relative to the ultrasound probe;
determining, by the controller and based on receipt of a response to a subset of the plurality of beams at a sensor at a location on the interventional medical device, the combination of time of emission and angle of emission relative to the ultrasound probe of the one of the subset of the plurality of beams,
determining, by the processor, the orientation of the interventional medical device based on the time of emission and angle of emission relative to the ultrasound probe of the one of the subset of the plurality of beams, and
determining, by the processor, characteristics of a wave travelling down the interventional medical device (205) as the response to the subset of the plurality of beams,
wherein determining the orientation is additionally based on the characteristics of the wave travelling down the interventional medical device (205) as the response to the subset of the plurality of beams.

16. A system for determining an orientation of an interventional medical device, comprising:
a sensor at a location on an interventional medical device;
an ultrasound probe that emits a plurality of beams each at a different combination of time of emission and angle of emission relative to the ultrasound probe; and
a controller comprising a memory that stores instructions and a processor that executes the instructions,
wherein, when executed by the processor, the instructions cause the controller to execute a process comprising:
controlling emission by the ultrasound probe of the plurality of beams;
determining, based on receipt of a response to a subset of the plurality of beams at the sensor, the combination of time of emission and angle of emission relative to the ultrasound probe of the one of the subset of the plurality of beams, and
determining the orientation of the interventional medical device based on the time of emission and angle of emission relative to the ultrasound probe of the one of the subset of the plurality of beam, and
determining characteristics of a wave travelling down the interventional medical device as the response to the subset of the plurality of beams,
wherein determining the orientation is additionally based on the characteristics of the wave travelling down the interventional medical device as the response to the subset of the plurality of beams.
